(19) United States
(12) Patent Application Publication
(10) Pub. No.: US 2024/0306909 A1
(43) Pub. Date: Sep. 19, 2024

(54) AUTOMATIC TONOMETER

(71) Applicant: Dmytro Viktorovych VAKULENKO, Ternopil (UA)

(72) Inventor: Dmytro Viktorovych VAKULENKO, Ternopil (UA)

(21) Appl. No.: 18/258,565

(22) PCT Filed: Jan. 14, 2022
(86) PCT No.: PCT/UA2022/000001

§ 371 Date: Jun. 21, 2023

(30) Foreign Application Priority Data
Nov. 18, 2021 (UA) U 2021 06521

(51) Int. Cl. A61B 3/16; A61B 5/00; A61B 5/0205

(52) U.S. Cl. CPC A61B 3/16; A61B 5/0205; A61B 5/742

(57) ABSTRACT

The claimed invention relates to the field of medicine, namely to oscillometric means of measuring blood pressure. The automatic tonometer contains a pneumatic cuff with valves (not marked in the figure), pneumatically connected by a hose with a pressure sensor that converts the change in pressure in the pneumatic cuff 1 into an electrical signal, the output of which is connected to the input of the processor through an analogue-to-digital converter that processes the electrical signal, the output of which is connected to the display, which displays the signal in the form of mathematical symbols, graphs, diagrams, according to which blood pressure parameters are set. The proposed automatic tonometer provides an expansion of the functional possibilities of the tonometer application and will allow building an expert system for differential diagnosis of diseases not only of the cardiovascular, but also the nervous and pulmonary systems.

### TECHNICAL FIELD

The claimed invention relates to the field of medicine, namely to oscillometric means of measuring blood pressure.

### PRIOR ART

From the existing state of technology, which relates to the analysed area, the closest, in terms of a set of features, to the claimed utility model is an automatic tonometer, which contains a pneumatic cuff with valves, pneumatically connected to a pressure sensor that converts a pneumatic signal into an electrical one, the output of which is through an analogue-to-digital converter with the input of a processor that processes the electrical signal, the output of which is connected to a display that displays the signal in the form of mathematical symbols, which are used to set blood pressure parameters (https://technosova.ru/dlja-zdorovja/tonometr/podkachivaet-vozduh-novtorno/)

### BACKGROUND OF THE INVENTION

The claimed invention is similar to the known tonometer in the following set of features: it contains a pneumatic cuff that is pneumatically connected to a pressure sensor, the output of which is connected to an analogue-to-digital converter, the output of which is connected with a display through a processor.

However, the known tonometer does not provide the technical result of the invention, which is due to the capabilities of the processor that processes the electrical signal from the pressure sensor, and ensures the establishment of the upper and lower arterial pressure as well as heart rate, that is, it has a low clinical and diagnostic characteristic that does not provide an assessment of the adaptation capabilities of various parts of the body to compression of the shoulder (another part of the body, human and animal) and with their help, determine the indicator of the activity of regulatory systems (IARS.AO), integrated functional vascular potential (IFVP), evaluate adaptation potential when performing functional tests (shoulder compression, Ruffier test), which will allow performing differential diagnosis of the cardiovascular, nervous and pulmonary systems diseases.

The task to be solved by the proposed claimed invention is to improve the known tonometer by changing the processor that processes the electrical signal, which reflects the change in pressure in the cuff that will ensure the expansion of the functional capabilities of the tonometer use and allow the construction of an expert system for the differential diagnosis of diseases not only of cardiovascular, but also nervous and pulmonary systems.

### DESCRIPTION

The set task is solved in a tonometer, containing a pneumatic cuff that is pneumatically connected to a pressure sensor, the output of which is connected to an analogue-to-digital converter, the output of which is connected through a processor to a display that, according to the object of the invention, uses a processor with an algorithm

$${f}_{ao} ( x ) \cdot {f}_{t} ( x ) \cdot {f}_{sp} ( x ) \cdot {f}_{m} ( x ) \cdot {f}_{op} ( x ) \cdot {f}_{fr} ( x ) = y ( x ) ,$$

Where the matrices:
fₐₒ(x)—values of the electrical signal registered from the sensor, including arterial oscillations;
fₜ(x)—values of indicators by the time method of analysis;
fₛₚ(x)—values of indicators by the spectral method of analysis;
fₘ(x)—values of indicators by the morphological method of analysis;
f_{fr}(x)—values of indicators by the fractal method of analysis;
fₒₚ(x)—values of indicators obtained from the ratio of interval and amplitude components;
y(x)—an integral matrix, including the values and functions of the matrices listed above.

### SUMMARY OF THE INVENTION

The proposed invention, in the scope of the specified set of features, provides a technical result, which consists in expanding the functional capabilities of the tonometer use and will allow the building of an expert system for the differential diagnosis of diseases not only of the cardiovascular, but also the nervous and pulmonary systems due to the fact that the analysis of values of the electrical signal registered from the sensor, including arterial oscillations during cuff compression of the research area fₐₒ(x) is carried out as well as calculating matrix indicators used in electrocardiography (heart rate variability (with adaptation)) by the time method of analysis fₜ(x) and the spectral one fₛₚ(x)—calculating the power spectrum (Fourier and Hilbert-Huang transforms). To conduct a spectral analysis in AO, depending on the degree of compression, there are three (from registered oscillations at the minimum pressure in the cuff to reaching diastolic pressure (APd), between APd and systolic pressure (APs), from APs to maximum compression) and five its parts (from the registered oscillations at the minimum pressure in the cuff to reaching APd, from the appearance of APd to 70% of the amplitude of the pulsations, from 70% to 100% of the amplitudes, from 100% to the appearance of APs, from the appearance of APs to maximum compression), fractal method of analysis to the arterial oscillogram in different phases of compression, in general, and to its intervalograms f_{fr}(x), to the arterial oscillogram and the ratio of interval and amplitude components—the optimal method of analysis fₒₚ(x) of the arterial oscillogram. The objects of study at morphological analysis are the general shape of the oscillogram and the nature of individual pulsations in different phases of compression, which are formed into a vector matrix of morphological indicators fₘ(x). A biological interpretation of indicators is carried out for the aggregate matrix of the vector and a number of complex indicators are calculated. Indicator of the activity of the regulatory systems of the Arterial oscillogram (IARS-AO), Indicator of the activity of the regulatory systems of the Arterial oscillogram based on the analysis of the intervalogram (IARS-AOI), Integrated functional vascular potential (IFVP), Index of the vegetative cardiac factor of the hemodynamics (IVCFH), Index of the centralization of the cardiac factor of the hemodynamics (ICCFH), Index of the vegetative vascular factor of hemodynamics (IVVFH), Index of centralization of the vascular factor of hemodynamics (ICVFH)

$${f}_{ao} ( x ) \cdot {f}_{t} ( x ) \cdot {f}_{sp} ( x ) \cdot {f}_{m} ( x ) \cdot {f}_{op} ( x ) \cdot {f}_{fr} ( x ) = y ( x )$$

According to the result of applying the indicated methods fₜ(x)·fₛₚ(x)·fₘ(x)·fₒₚ(x)·f_{fr}(x) of analysis to the signal fₐₒ(x) a matrix is obtained y(x), which is represented by indicators, semantic values and functions. To the obtained vector y(x) machine learning methods—a classification task Z_{cl}(y), methods of regression modelling Zᵣ(y), cluster analysis Z_{cla}(y) and semantic analysis Zₛₑₘ(y) are used. According to the results of the received matrix of indicators and judgments Z_{cl}(y)·Zᵣ(y)·Z_{cla}(y)·Zₛₑₘ(y) a matrix of user states is formed Exp(n) depending on his/her specialty.

$${Z}_{cl} ( y ) \cdot {Z}_{r} ( y ) \cdot {Z}_{cla} ( y ) \cdot {Z}_{sem} ( y ) = Exp ( n )$$

### BRIEF DESCRIPTION OF DRAWINGS

The proposed automatic tonometer is explained by the drawings, which are given in:

FIG. 1—a schematic diagram of an automatic tonometer

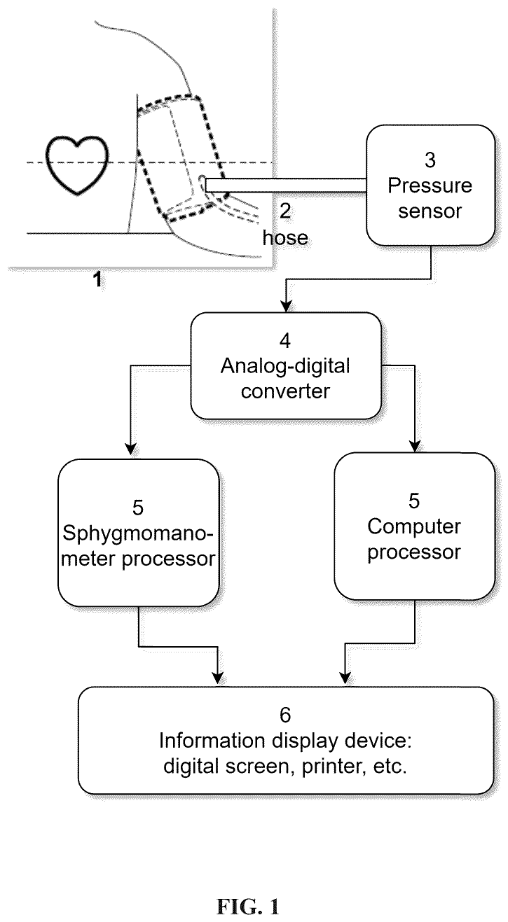

FIG. 2—the work scheme of the processor of the automatic tonometer.

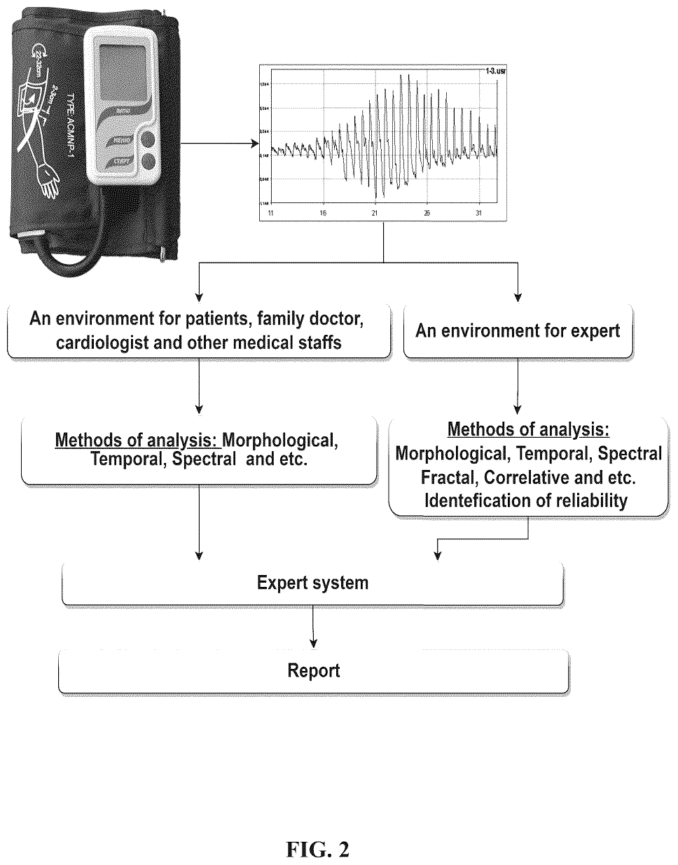

### DETAILED DESCRIPTION OF THE INVENTION

The proposed automatic tonometer contains a pneumatic cuff 1 with valves (not marked in the figure), pneumatically connected by a hose 2 to a pressure sensor 3, which converts the change in pressure in the pneumatic cuff 1 into an electrical signal, the output of which is through an analogue-to-digital converter 4 connected to the input of the processor 5 that processes the electrical signal, the output of which is connected to the display 6 that displays the signal in the form of mathematical symbols, graphs, diagrams, according to which the blood pressure parameters of the object (person, animal) are set.

The proposed automatic tonometer will be used in the following way.

The area of the human (or animal) body, depending on the research, for example, the area of the shoulder, is covered with a pneumatic cuff 1 and fixed, after which air is pumped into the cuff, in response to compression of the shoulder by the cuff, pulsations occur in the cuff caused by the activity of the vascular wall during the movement of the blood flow, the specified pulse activity causes a change in the pressure in the cuff (registration can be carried out during compression, decompression or during a period of constant pressure), which is pneumatically connected to a pressure sensor that converts a pneumatic signal into an electrical one, the output of which is through an analogue-to-digital converter with a processor input that processes an electrical signal from which an arterial oscillogram fao(x) is extracted, to which Arterial oscillography methods are applied and an array of vector matrices is obtained according to time ft(x)=[pNN50_pos=8.1, SDSD_pos=0.18, Mpos=0.8, AMpos=36, BP_pos=0.72, IVR_pos=44.42, VPR_pos=1.07, IN_pos=28, HVR_index_pos=48.66, NN50_neg=9, SDSD_neg=0.27, Mneg=0.8, AMneg=34.68, BP_neg=1, IVR_neg=30.56, VPR_neg=1.31, IN_neg=20.43, HVR_index_neg=46.69, . . . ] Morphological M1=5.06, M2=3.05, M3=1.40, M4=1.00, M5=4.35, M6=1.90, M7=3.08, M8=4.10, M9=3.13, Mcp=3.12, . . . spectral fsp(x)=[ULF_per=2.96, VLF_per=11.71, LF_per=21.44, HF_per=59.59, ULF_per_int_p 6.68, VLF_per_int_p=11.9, LF_per_int_p=30.05,HF_per_int_p=59.59, ULF_per_int_n=6.68, VLF_per_int_n=11.9, LF_per_int_n=30.05, HF_per_int_n=49.67, Delta_per=98.21, Teta_per=1.51, Alpha_per=0.16, Beta_per=0.09, ULF_per=2.96, ULF_per_20=0.88, ULF_per_20-70=2.86,ULF_per_70-100=6.06, ULF_per_100-70=5.06, ULF_per_70-end=3.0, VLF_per=11.71, LF_per_20=26.25, VLF_per_20-70=16.47, VLF_per_70-100=23.51, VLF_per_100-7015.64, VLF_per_70-end=14.06, LF_per=21.44, LF_per_20=26.25, LF_per_20-70=29.66, LF_per_70-100=31.06, LF_per_100-70=21.39, LF_per_70-end=24.75, HF_per=59.59, HF_per_20=65.47, HF_per_20−70=47.92, HF_per_70-100=36.55, HF_per_100-70=47.21,HF_per_70-end=54.07, ULF_per_int_p=6.68, LF_per_20_int_p=0.1, ULF_per_20-70_int_p=0.12, ULF_per_70-100_int_p=0.06, ULF_per_100-70_int_p=0.02, ULF_per_70-end_int_p=6.9, VLF_per_int_p=11.9, VLF_per_20_int_p=0.01, VLF_per_20-70_int_p=0.01, VLF_per_70-100_int_p=0.49, VLF_per_100-70_int_p=0.01, VLF_per_70-end_int_p=11.87, LF_per_int_p=30.05, LF_per_20_int_p=0.01, LF_per_20-70_int_p=0.02, LF_per_70-100_int_p=5.22, LF_per_100-70_int_p, LF_per_70-end_int_p, LF_per_70-100_int_p=5.22, LF_per_100-70_int_p=0.01, LF_per_70-end_int_p=27.17, HF_per_int_p=49.67, HF_per_20_int_p=0.01, HF_per_20-70_int_p=0.02, HF_per_70-100_int_p=12.50, HF_per_100-70_int_p=0.02, HF_per_70-end_int_p=48.33, ULF_per_int_n=4.39, ULF_per_20_int_n=0.01, ULF_per_20-70_int_n=0.88, ULF_per_70-100_int_n=2.96, ULF_per_100-70_int_n=0.01, ULF_per_70-end_int_n=3.69, VLF_per_int_n=9.69, VLF_per_20_int_n=0.01, VLF_per_20-70_int_n=3.41, VLF_per_70-100_int_n=5.04, VLF_per_100-70_int_n=0.01, VLF_per_70-end_int_n=7.92, LF_per_int_n=25.79, LF_per_20_int_n=0.01, LF_per_20-70_int_n=15.43, LF_per_70-100_int_n,20.49, LF_per_100-70_int_n=0.01, LF_per_70-end_int_n=24.77, HF_per_int_n=56.58, HF_per_20_int_n=0.01, HF_per_20-70_int_n=40.78, HF_per_70-100_int_n=25.00, HF_per_100-70_int_n=0.00, HF_per_70-end_int_n=59.63, Delta_per=98.21, Delta_per_20=97.71, Delta_per_20-70=94.13, Delta_per_70-100=96.85, Delta_per_100-70=93.87, Delta_per_70-end=92.17, Teta_per=1.51, Teta_per_20=1.37, Teta_per_20-70=4.16, Teta_per_70-100=2.27, Teta_per_100-70=3.74, Teta_per_70-end=5.81, Alpha_per=0.16, Alpha_per_20=0.40, Alpha_per_20-70=0.65, Alpha_per_70-100=0.44, Alpha_per_100-70=0.79, Alpha_per_70-end=0.94, Beta_per=0.09, Beta_per_20=0.38, Beta_per_20-70=0.34, Beta_per_70-100=0.32, Beta_per_100-70=0.60, Beta_per_70-end=0.74, . . . ]. fractal ffr(x)=[0.6, 0.52, 0.45, 0.52, 0.61, 0.64, . . . ], optimal fop(x)=[1.2, 1.4, 10.6, 0.7, 0.8, 0.17, . . . ] To the formed matrix y(x) calculated complex fc(x) indicators are added IARS-AO=3—moderate functional stress, IARS-AOI=4—moderate functional stress and IFVP=4—pre-nozological condition, a matrix of conditions is formed, IVCFH=2 Normotonia, Vegetative balance (IVCFH=−2-+2), ICCFH=1 Vegetative regulation (ICCFH=1, . . . , −1), IVVFH=2 Moderate sympathicotonia. (IVVFH=+3-+5), ICVFH=1 Vegetative regulation (ICVFH=1, . . . , −1) fs(x)=G of the examined person fao(x)ft(x)fsp(x)fm(x)fop(x)ffr(x)fc(x)fs(x)fruf(x) . . . =y(x)

Based on the result of applying the specified ft(x)fsp(x)fm(x)fop(x)ffr(x) analysis methods to the signal fao(x), a matrix-vector y(x) is obtained, which is represented by indicators, semantic expressions and functions. Machine learning methods are applied to the obtained vector y(x)—the classification problem Zcl(y)=[Cardiovascular diseases—38%, Arterial hypertension AH-10%, Arterial hypertension AH-20%, Diseases of the pulmonary system—9%, Mental (neurological) diseases—3%, Arrhythmia—17%, COVID-19-12%, regression modeling Zr(y)=blood indicators (Blood sugar=5, Endothelin=4, . . . , ), dynamic state of Endothelium=6, Level of Anxiety ±12%, Depression ±6%, Indicators of Central and Peripheral Hemodynamics (Time of propagation of an elastic wave=0.088, Tonus of big artery=0.18, Stroke volume of blood=63.3, Total peripheral resistance=1023, . . . ), cluster analysis Zcla(y)=[PcULF_per=12, PcAlpha_per_20=10, PcTeta_per=7, Pi70-end=17, . . . ] and semantic analysis Zsem(y)=[Id=6, It=4, . . . ,]. Based on the results of the received matrix of indicators and judgments Zcl(y)Zr(y)Zcla(y)Zsem(y), the Expert system forms a matrix of patient states Exp(n).

$$Zcl ( y ) Zr ( y ) Zcla ( y ) Zsem ( y ) … = Exp ( n )$$

Depending on the user's specialty, the Expert System forms a matrix of patient states Exp(n)

For the patient Exp(n)=maintain a healthy lifestyle. For the family doctor, a report based on the matrix Zcl(y)=[Cardiovascular diseases—38%, Arterial hypertension AH-10%, Arterial hypertension AH-20%, Diseases of the pulmonary system—9%, Mental (neurological) diseases—3%, Arrhythmia—17%, COVID-19-12%, regression modeling Zr(y)=blood indicators (Blood sugar=5, Endothelin=4, . . . , ), dynamic state of Endothelium=6, Alarm Level ±12%, Depression ±6%, Indicators of Central and Peripheral Hemodynamics (Time of propagation of an elastic wave=0.088, Tonus of big artery=0.18, Stroke volume of blood=63.3, Total Peripheral Resistance=1023, . . . ), cluster analysis Zcla(y)=[PcULF_per=12, PcAlpha_per_20=10, PcTeta_per=7, Pi70-end=17, . . . ] and semantic analysis Zsem(y)=[Id=6, It=4, . . . ,].

For a cardiologist: the content of the matrices y(x), Zcl(y)Zr(y)Zcla(y)Zsem(y) is proposed.

For a pediatrician for admission to physical education classes: the content of the Zcl(y) and fruf(x) matrices is proposed.

Processor 5, which works according to the specified algorithm, can be installed both in tonometers and be external—a personal computer processor is used, which is connected to the analogue-to-digital converter 4 of the tonometer.

What is claimed is:

1. A tonometer containing a pneumatic cuff that is pneumatically connected to a pressure sensor, the output of which is connected to an analogue-to-digital converter, the output of which is connected through a processor with a display that differs in that it uses a processor with an algorithm:
$${f}_{ao} ( x ) \cdot {f}_{t} ( x ) \cdot {f}_{sp} ( x ) \cdot {f}_{m} ( x ) \cdot {f}_{op} ( x ) \cdot {f}_{fr} ( x ) = y ( x ) ,$$
Where the matrices:
fₐₒ(x)fₐₒ(x)—values of the electrical signal registered from the sensor, including arterial oscillations;
fₜ(x)fₜ(x)—values of indicators by the time method of analysis;
fₛₚ(x)fₛₚ(x)—values of indicators by the spectral method of analysis;
fₘ(x)fₘ(x)—values of indicators by the morphological method of analysis;
f_{fr}(x)f_{fr}(x)—values of indicators by the fractal method of analysis;
fₒₚ(x)fₒₚ(x)—values of indicators obtained from the ratio of interval and amplitude components;
y(x)y(x)—an integral matrix, including the values and functions of the matrices listed above.
With further formation of the states matrix Exp(n) of the user according to the following algorithm:
$${Z}_{cl} ( y ) \cdot {Z}_{r} ( y ) \cdot {Z}_{cla} ( y ) \cdot {Z}_{sem} ( y ) = Exp ( n )$$
Where the matrices:
Z_{cl}(y)—values and functions based on machine learning methods—a classification task;
Zᵣ(y)—values and functions based on regression modelling methods;
Z_{cla}(y)Z_{cl}(y)—values and functions based on methods of cluster analysis;
Zₛₑₘ(y)—values and functions based on methods of semantic analysis;
